(19) United States
(12) Patent Application Publication
(10) Pub. No.: US 2018/0142297 A1
(43) Pub. Date: May 24, 2018

(54) SYSTEMS AND METHODS FOR CHARACTERIZING GRANULOMATOUS DISEASES

(71) Applicant: ARIZONA BOARD OF REGENTS ON BEHALF OF THE UNIVERSITY OF ARIZONA, TUCSON, AZ (US)

(72) Inventors: Joe GN GARCIA, Tucson, AZ (US); Tong Zhou, Tucson, AZ (US)

(21) Appl. No.: 15/573,605

(22) PCT Filed: May 13, 2016
(86) PCT No.: PCT/US16/32300

§ 371 Date: Nov. 13, 2017

Related U.S. Application Data
(60) Provisional application No. 62/161,521, filed on May 14, 2015.

(51) Int. Cl. C12Q 1/6883

(52) U.S. Cl. CPC C12Q 1/6883; C12Q 2600/178; C12Q 2600/158

(57) ABSTRACT

The present disclosure relates to methods of characterizing disease. In particular, the present disclosure relates to genes associated with complicated sarcodosis.

### CROSS REFERENCE TO RELATED APPLICATIONS

This application claims the benefit of U.S. provisional application Ser. No. 62/161,521, filed May 14, 2015, which is incorporated herein by reference in its entirety.

### FIELD OF THE INVENTION

The present disclosure relates to methods of characterizing disease. In particular, the present disclosure relates to genes associated with complicated sarcodosis.

### BACKGROUND OF THE INVENTION

Sarcoidosis is a systemic inflammatory heterogeneous disease characterized by the presence of non-caseating epithelioid granulomas in one or multiple organs. Lungs are commonly affected in about 90% of the cases, commonly manifested as bilateral hilar lymphadenopathy (BHL) and pulmonary infiltration and in some cases pulmonary fibrosis. Ocular and skin lesions may be present as well and organs such as the liver, spleen, lymph nodes, salivary glands, heart, nervous system, muscles, bones may also be involved (Iannuzzi M C, et al., N Engl J Med 2007, 357:2153-2165; Newman L S, et al., N Engl J Med 1997, 336:1224-1234). Lofgren syndrome, an acute presentation of sarcoidosis, is characterized by erythema nodosum, BHL, and polyarthralgia; it is associated with good prognosis, the majority of the active patients may have spontaneous regression (Byun C W, et al., Ann Rehabil Med 2013, 37:295-299). Sarcoidosis diagnosis, according to the criteria of the World Association of Sarcoidosis and Granulomatous Diseases (WASOG), is based on typical clinical features together with granulomas on lung biopsies (Am J Respir Crit Care Med 1999, 160:736-755). Due to the wide spectrum of manifestations, diagnosis is challenging as it may mimic multiple rheumatologic illness (Sweiss N J, et al., Semin Respir Crit Care Med 2010, 31:463-473; Drent M, et al., Curr Opin Rheumatol 2014, 26:276-284).

Though sarcoidosis may be asymptomatic and/or chronic, some patients may develop complications with gradual damage to vital organs including the heart. In addition, significant racial and gender differences in disease development and prognosis have also been reported (highest annual incidence in African American females) (Rybicki B A, et al., Am J Epidemiol 1997, 145:234-241), Irish and Scandinavian populations. Furthermore, some sarcoidosis patients may recover completely and the disease may improve or clear up spontaneously; yet, more than 50% of will experience remission within 3 years after diagnosis, and two thirds of the patients have remission within 10 years (Nunes H, et al., Orphanet J Rare Dis 2007).

Despite significant advances, the etiology of sarcoidosis still remains unclear. Data indicates linkage with some mycobacterial and propionibacterial organism in some studies. There is no consensus on the nature of a microbial pathogenesis of sarcoidosis and environmental factors (e.g., mold/mildew exposure) (Rossman MD, et al., Am J Hum Genet 2003, 73:720-735). Similarly, hyperimmune TH1 response has been postulated (Chen E S, et al., Clinics in Chest Medicine 2008, 29:365; Saidha S, et al., Yale Journal of Biology and Medicine 2012, 85:133-141). Though the candidate gene approach (e.g., studies focusing on immunopathogenesis) and epidemiological studies have begun to illustrate the abnormal gene expression (Vissinga C, et al., Hum Immunol 1996, 48:98-106; Grunewald J, et al., Am J Respir Crit Care Med 1995, 151:151-156) and genetic variation (e.g., specific HLA genotypes) (Iannuzzi et al., supra; Newman et al., supra; Brewerton D A, et al., Clin Exp Immunol 1977, 27:227-229; Rossman M D, et al., Am J Hum Genet 2003, 73:720-735), that may contribute to sarcoidosis, given its high heterogeneity in clinical course and severity, a significant challenge remains to elucidate comprehensively the pathogenesis of sarcoidosis and the elements that elicit remission versus chronicity or the multiple organ manifestations, as well as the population/ethnic differences influence on pathogenesis, and prognosis.

Due to the lack of understanding of the natural course of the disease, there is no consensus on the right time to initiate therapy and for how long should patients be treated to avoid progression; the therapeutic approach is limited to treating the clinical manifestations. Topical or systemic glucocorticosteroids are used as a first line therapy. Almost half of patients do not require long systemic therapy and in cases of Lofgren's NSAIDs are included. Second-line therapy includes disease-modifying antisarcoid drugs (DSMASDs). Recently B-cell targeted therapies with chimeric monoclonal antibodies including anti-TNF-[alpha] and CD-20 targeting, have been approached for refractory sarcoidosis with variable results (Drent M, et al., Curr Opin Rheumatol 2014, 26:276-284; Sweiss N J, et al., In Eur Respir J. Volume 43. 2014: 1525-1528) and considerable toxicities. Corticosteroid therapy has not been demonstrated to improve lung function, prevent fibrosis or disease progression (Paramothayan N S, et al., Cochrane Database Syst Rev 2005) in the long term.

As such, for better management of sarcoidosis, diagnostic and prognostic biomarkers are needed to identify patients at high risk for future development of complicated sarcoidosis (Zhou T, et al., PLoS One 2012).

### SUMMARY OF THE INVENTION

The present disclosure relates to methods of characterizing disease. In particular, the present disclosure relates to genes associated with complicated sarcodosis.

Additional embodiments provide a method of identifying miRNA expression associated with sarcodosis and/or complicated sarcodosis, comprising: a) assaying a sample from a subject for the presence of altered expression of one or more (e.g., 1, 2, 3, 4, 5, 6, 7, or all) miRNAs selected from miR-23a, miR-23b, miR-30c, miR-93, miR-143, miR-185, miR-196a, or miR-223 and/or altered gene expression of one or more (e.g., 1, 2, 3, 4, 5, 6, 7, 8, 9, 10, 11, 12, 13, 14, 15, 16, or all) genes selected from ADORA3, ATP10A, CBLB, EFHA2, ERCC6L2, FIGNL1, GALNT12, IL6ST, ITGA6, MBTPS1, MTERFD2, SATB1, SORCS3, STAT4, TMEM263, URI11, or ZFYVE9; and b) identifying the subject as having sarcodosis or complicated sarcodosis based on the presence of the altered miRNA and/or gene expression.

In some embodiments, the method further comprises the step of calculating a diagnostic score for the sample based on the expression of the one or more genes. In some embodiments, a higher positive number score is indicative of the subject having or being at risk for complicated sarcoidosis. In some embodiments, the sample is tissue, blood, plasma, serum, or lung cells. In some embodiments, the detecting comprises forming a complex between the genes and/or miRNAs and a nucleic acid primer, probe, or pair of primers that specifically bind to the genes and/or miRNAs.

Some embodiments provide at least two complexes comprising a nucleic acid ncoding a miRNA selected from two or more (e.g., 2, 3, 4, 5, 6, 7, or all) miRNAs selected from miR-23a, miR-23b, miR-30c, miR-93, miR-143, miR-185, miR-196a, or miR-223; and at least two distinct nucleic acid primers or probes that specifically hybridize to the two or more miRNAs and/or at least two complexes comprising a nucleic acid encoding a gene selected from two or more (e.g., 2, 3, 4, 5, 6, 7, 8, 9, 10, 11, 12, 13, 14, 15, 16, or all) genes selected from ADORA3, ATP10A, CBLB, EFHA2, ERCC6L2, FIGNL1, GALNT12, IL6ST, ITGA6, MBTPS1, MTERFD2, SATB1, SORCS3, STAT4, TMEA1263, UR11, or ZFYVE9; and at least two distinct nucleic acid primers or probes that specifically hybridize to the two or more genes.

Still other embodiments provide kits or systems, comprising: reagents for detecting altered expression levels of two or more (e.g., 2, 3, 4, 5, 6, 7, or all) miRNAs selected from miR-23a, miR-23b, miR-30c, miR-93, miR-143, miR-185, miR-196a, or miR-223 and/or reagents for detecting altered gene expression levels of two or more (e.g., 2, 3, 4, 5, 6, 7, 8, 9, 10, 11, 12, 13, 14, 15, 16, or all) genes selected from ADORA3, ATP10A, CBLB, EFHA2, ERCC6L2, FIGNL1, GALNT12, IL6ST, ITGA6, MBTPS1, MTERFD2, SATB1, SORCS3, STAT4, TMEM263, UR11 , or ZFYVE9.

In some embodiments, systems comprise software for determining a diagnosis or increasd risk of sarcodosis or complicated sarcodosis. In some embodiments, the reagents are at least two nucleic acid primers or probes that specifically hybridize to the two or more genes and/or miRNAs. In some embodiments, the nucliec acid primers or probes are at least 8 nucleic acids in length (e.g., at least 10 or at least 20).

Further embodiments provide a method of identifying miRNA expression, comprising: assaying a sample from a subject for the presence of altered expression of one or more (e.g., 1, 2, 3, 4, 5, 6, 7, or all) miRNAs selected from, for example, miR-23a, miR-23b, miR-30c, miR-93, miR-143, miR-185, miR-196a, or miR-223.

Additional embodiments provde a method of identifying gene expression, comprising: assaying a sample from a subject for the presence of altered gene expression of one or more (e.g., 1, 2, 3, 4, 5, 6, 7, 8, 9, 10, 11, 12, 13, 14, 15, 16, or all) genes selected from ADORA3, ATP10A, CBLB, EFHA2, ERCC6L2, FIGNL1, GALNT12, IL6ST, ITGA6, MBTPS1, MTERFD2, SATB1, SORCS3, STAT4, TMEM1263, UR11, or ZFYVE9.

Yet other embodiments provide a method of identifying gene expression, comprising: assaying a sample from a subject for the presence of altered expression of one or more (e.g., 1, 2, 3, 4, 5, 6, 7, or all) miRNAs selected from, for example, miR-23a, miR-23b, miR-30c, miR-93, miR-143, miR-185, miR-196a, or miR-223; and assaying a sample from a subject for the presence of altered gene expression of one or more (e.g., 1, 2, 3, 4, 5, 6, 7, 8, 9, 10, 11, 12, 13, 14, 15, 16, or all) genes selected from the group consisting of ADORA3, ATP10A, CBLB, EFHA2, ERCC6L2, FIGNL1, GALNT12, IL6ST, ITGA6, MBTPS1, MTERFD2, SATB1, SORCS3, STAT4, TMEA1263, UR11, or ZFYVE9. In some embodiments, the assaying comprises the use of a reagent selected from, for example, one or more nucleic acid probes that specifically hybridize to the genes or miRNAs, a pair of amplification primers that specifically hybridizes to the genes or miRNAs, or one or more sequencing primers that specifically hybridize to the genes or miRNAs.

Additional embodiments are described herein.

### DESCRIPTION OF THE FIGURES

FIG. 1 shows an exemplary 8-miRNA signature associated with sarcodosis.

FIG. 2 shows an exemplary 17-gene signature associated with complicated sacrodosis.

FIG. 3 shows the performance of the 17-gene signature in the validation cohorts.

FIG. 4 shows a boxplot of severity score for the subjects with sarcodosis.

### DEFINITIONS

To facilitate an understanding of the present invention, a number of terms and phrases are defined below:

As used herein, the terms “detect”, “detecting” or “detection” may describe either the general act of discovering or discerning or the specific observation of a detectably labeled composition.

As used herein, the term “subject” refers to any organisms that are screened using the diagnostic methods described herein. Such organisms preferably include, but are not limited to, mammals (e.g., humans).

The term “diagnosed,” as used herein, refers to the recognition of a disease by its signs and symptoms, or genetic analysis, pathological analysis, histological analysis, and the like.

Fibrosis” means the formation or development of excess fibrous connective tissue in an organ or tissue. In certain embodiments, fibrosis occurs as a reparative or reactive process. In certain embodiments, fibrosis occurs in response to damage or injury. The term “fibrosis” is to be understood as the formation or development of excess fibrous connective tissue in an organ or tissue as a reparative or reactiv

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-i sopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues (e.g., biopsy samples), cells, and gases. Biological samples include blood products, such as plasma, serum and the like. Such examples are not however to be construed as limiting the sample types applicable to the present invention.

### DETAILED DESCRIPTION OF THE INVENTION

The present disclosure relates to methods of characterizing disease. In particular, the present disclosure relates to genes associated with complicated sarcodosis.

Accordingly, embodiments of the present disclosure provide research, sceening, diagnostic, and prognostic methods for characterizing fibrosis (e.g., diagnosing sarcodosis or distinguishing between complicated and uncomplicated sarcodosis).

### I. Diagnostic and Screening Methods

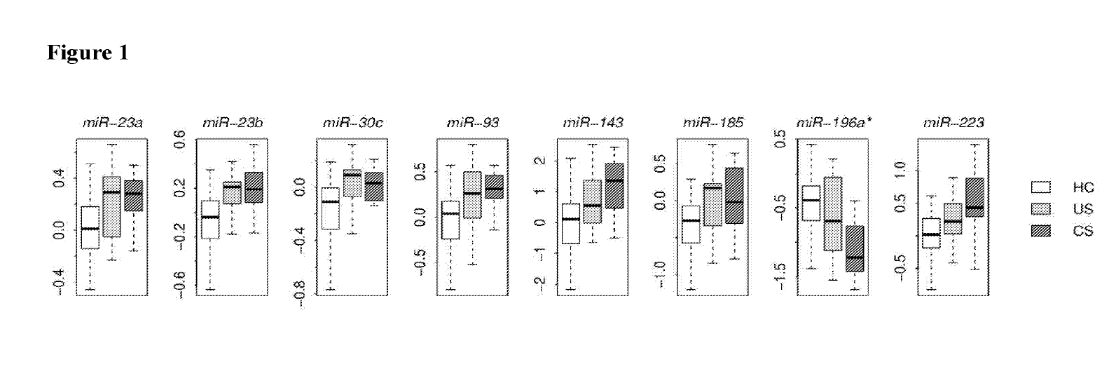

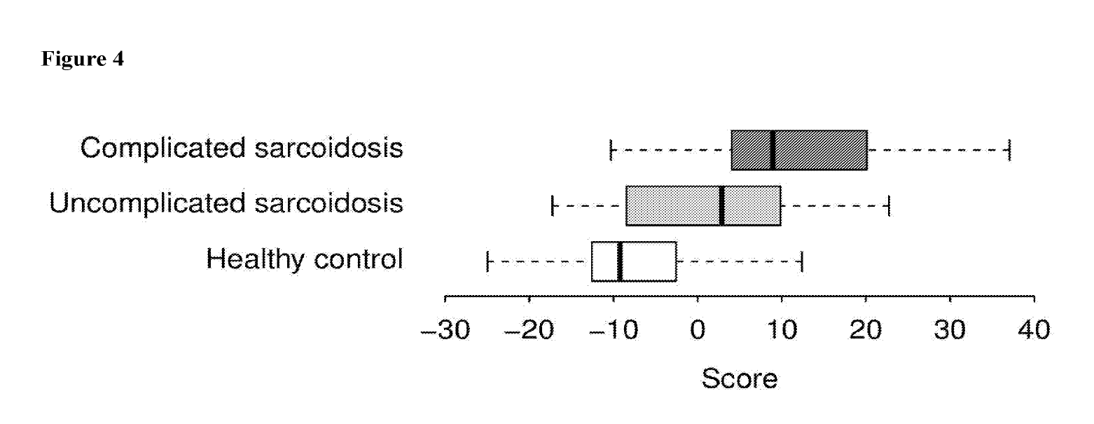

As described above, embodiments of the present invention provide diagnostic and screening methods that utilize the detection of altered gene expression levels of one or more genes and/or miRNAs (e.g., 1, 2, 3, 4, 5, 6, 7, 8, 9, 10, 11, 12, 13, 14, 15, 16, 17, 18, 19, 20, or all) of those shown in Table 1 and FIGS. 1-4 (e.g., miR-23a, miR-23b, miR-30c, miR-93, miR-143, miR-185, miR-196a, or miR-223); or (ADORA3, ATP10A, CBLB, EFHA2, ERCC6L2, FIGNL1, GALNT12, IL6ST, ITGA6, MBTPS1, MTERFD2, SATB1, SORCS3, STAT4, TMEM263, URI1, or ZFYVE9). Exemplary, non-limiting methods are described below.

In some embodiments, the presence of altered expression of one or more of the miRNAs and/or genes described herein are indicative of a diagnosis, characterization, or increased risk of sarcodosis. In some embodiments, altered expression of one or more of the genes described herein are indicative of a diagnosis, characterization, or increased risk of complicated sarcodosis.

Any patient sample suspected of containing the genes and/or miRNAs may be tested according to methods of embodiments of the present invention. By way of non-limiting examples, the sample may be tissue (e.g., a lung biopsy sample), blood, urine, or a fraction thereof (e.g., plasma, serum, cells).

In some embodiments, the patient sample is subjected to preliminary processing designed to isolate or enrich the sample for the genes or cells that contain the gene. A variety of techniques known to those of ordinary skill in the art may be used for this purpose, including but not limited to: centrifugation; immunocapture; cell lysis; and, nucleic acid target capture (See, e.g., EP Pat. No. 1 409 727, herein incorporated by reference in its entirety).

In some embodiments, expression levels of the genes are detected along with other markers in a multiplex or panel format. Markers are selected for their predictive value alone or in combination with the levels of genes expression. Markers for other diseases, infections, and metabolic conditions are also contemplated for inclusion in a multiplex or panel format.

i. DNA and RNA Detection

The levels of gene expression of the genes and/or miRNAs described herein are detected using a variety of nucleic acid techniques known to those of ordinary skill in the art, including but not limited to: nucleic acid sequencing; nucleic acid hybridization; and, nucleic acid amplification.

1. Sequencing

A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 250 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing (see, e.g., Astier et al., J. Am. Chem. Soc. 2006 Feb. 8; 128(5):1705-10, herein incorporated by reference) is utilized. The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, HeliScope by Helicos BioSciences (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety) is utilized. Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb to 100 Gb generated per run. The read-length is 100-300 base pairs. The accuracy for homopolymer repeats of 5 repeats in length is ˜98%. The benefits of ion semiconductor sequencing are rapid sequencing speed and low upfront and operating costs.

Stratos Genomics, Inc. sequencing involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “High Throughput Nucleic Acid Sequencing by Expansion,” filed June 19, 2008, which is incorporated herein in its entirety.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671956; U.S. patent application Ser. No. 11/781166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

2. Hybridization

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

In some embodiments, altered gene expression is detected using fluorescence in situ hybridization (FISH). In some embodiments, FISH assays utilize bacterial artificial chromosomes (BACs). These have been used extensively in the human genome sequencing project (see Nature 409: 953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

The present invention further provides a method of performing a FISH assay on human cells (e.g., breast or endometrial cells). Specific protocols are well known in the art and can be readily adapted for the present invention. Guidance regarding methodology may be obtained from many references including: In situ Hybridization: Medical Applications (eds. G. R. Coulton and J. de Belleroche), Kluwer Academic Publishers, Boston (1992); In situ Hybridization: In Neurobiology; Advances in Methodology (eds. J. H. Eberwine, K. L. Valentino, and J. D. Barchas), Oxford University Press Inc., England (1994); In situ Hybridization: A Practical Approach (ed. D. G. Wilkinson), Oxford University Press Inc., England (1992)); Kuo, et al., Am. J. Hum. Genet. 49:112-119 (1991); Klinger, et al., Am. J. Hum. Genet. 51:55-65 (1992); and Ward, et al., Am. J. Hum. Genet. 52:854-865 (1993)). There are also kits that are commercially available and that provide protocols for performing FISH assays (available from e.g., Oncor, Inc., Gaithersburg, Md.). Patents providing guidance on methodology include U.S. 5,225,326; 5,545,524; 6,121,489 and 6,573,043. All of these references are hereby incorporated by reference in their entirety and may be used along with similar references in the art and with the information provided in the Examples section herein to establish procedural steps convenient for a particular laboratory.

3. Microarrays

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts (e.g., those described in table 1) by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

4. Amplification

Nucleic acids may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

5. Protein Detection

In some embodiments, altered levels gene expression are detected by detected altered levels of polypeptides encoded by the genes (e.g., using immunoassays or mass spectrometry).

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays. Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

Mass spectrometry has proven to be a valuable tool for the determination of molecular structures of molecules of many kinds, including biomolecules, and is widely practiced today. Purified proteins are digested with specific proteases (e.g. trypsin) and evaluated using mass spectrometry. Many alternative methods can also be used. For instance, either matrix-assisted laser desorption/ionization (MALDI) or electrospray ionization (ESI) mass spectrometric methods can be used. Furthermore, mass spectroscopy can be coupled with the use of two-dimensional gel electrophoretic separation of cellular proteins as an alternative to comprehensive pre-purification. Mass spectrometry can also be coupled with the use of peptide fingerprint database and various searching algorithms. Differences in post-translational modification, such as phosphorylation or glycosylation, can also be probed by coupling mass spectrometry with the use of various pretreatments such as with glycosylases and phosphatases. All of these methods are to be considered as part of this application.

In some embodiments, electrospray ionisation quadrupole mass spectrometry is utilized to detect polypeptide levels (See e.g., U.S. Patent 8,658,396; herein incorporated by reference in its entirety).

6. Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., gene expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., presence or absence of altered levels of gene expression of the genes in Tables 1 or 2) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

6. Compositions & Kits

Compositions for use in the diagnostic methods described herein include, but are not limited to, probes, amplification oligonucleotides, and the like. In some embodiments, kits include all components necessary, sufficient or useful for detecting the markers described herein (e.g., reagents, controls, instructions, etc.). The kits described herein find use in research, therapeutic, screening, and clinical applications.

The probe and antibody compositions of the present invention may also be provided in the form of an array.

In some embodiments, the present invention provides one or more nucleic acid probes or primers having 8 or more (e.g., 10 or more, 12 or more, 15 or more, 18 or more, etc.) nucleotides, and that specifically bind to nucleic acids encoding one or more of the genes or miRNAs in table 1. In some embodiments, the present invention provides an antibody that specifically binds to one or more of the genes or miRNAs in table 1.

Embodiments of the present invention provide complexes of two or more nucleic acids described in table 1 with nucleic acid primers or probes. In some embodiments, the present invention provides a multiplex (e.g., microarray) comprising reagents that binds to two or more nucliec acids described in table 1.

### EXPERIMENTAL

The following examples are provided in order to demonstrate and further illustrate certain preferred embodiments and aspects of the present invention and are not to be construed as limiting the scope thereof.

### EXAMPLE 1

Sarcoidosis is a granulomatous lung disorder of unknown cause with many systemic manifestations. The majority of individuals with sarcoidosis spontaneously achieve full remission (uncomplicated sarcoidosis). However, approximately 20% of affected individuals experience progressive disease with respiratory, cardiac or nervous system involvement (complicated sarcoidosis). Therefore, diagnostic tools and biomarkers are needed to identify the patients who are likely to develop complicated sarcoidosis. Peripheral blood mononuclear cell (PBMC) gene expression profiling provides an opportunity to explore potential molecular gene signatures involved in identification of individuals with either uncomplicated or complicated sarcoidosis.

The PBMC microRNA expression levels from 35 healthy controls, 17 patients with uncomplicated sarcoidosis, and 13 patients with complicated sarcoidosis were profiled using Exiqon miRCURY LNA™ microRNA Array. PBMC protein-coding gene expression level from 35 healthy controls, 17 patients with uncomplicated sarcoidosis, and 22 patients with complicated sarcoidosis were also profiled using Affymetrix GeneChip Human Exon 1.0 ST Array. Spearman's rank correlation test was used to identify the microRNAs/genes that were differentially expressed with severity (healthy control→uncomplicated sarcoidosis→complicated sarcoidosis).

Fourty six microRNAs (adjusted P<0.05) and 1,559 genes (adjusted P<0.0005) were identified that were differentially expressed with sarcoidosis severity. A list of gene targets for the 46 dysregulated microRNAs was identified using the in silico prediction provided by microrna, with filtering based on a stringent mirSVR score cutoff of -1.2. These predicted mRNA targets were intersected against the 1,559 differentially expressed genes, revealing a total of 19 microRNA-mRNA pairs. These 19 miRNA-mRNA pairs consisted of 17 unique protein-coding genes yielding a 17-gene signature. Pathway analysis on the 17-gene signature revealed the Jak-STAT signaling pathway as the most significantly represented pathway (P<0.05). A diagnostic score was assigned to each patient based on the expression of the 17-gene signature. A high score indicated a higher likelihood of complicated sarcoidosis. A significant increasing trend (P<0.001) in the diagnostic score from healthy control, uncomplicated sarcoidosis, to complicated sarcoidosis was observed. IT was also demonstrated that this microRNA regulated gene signature can differentiate sarcoidosis patients from healthy controls in two independent validation cohorts. The areas under the receiver operating characteristic (ROC) curve (AUC) were 0.876 and 0.913 for the two validation cohorts, respectively.

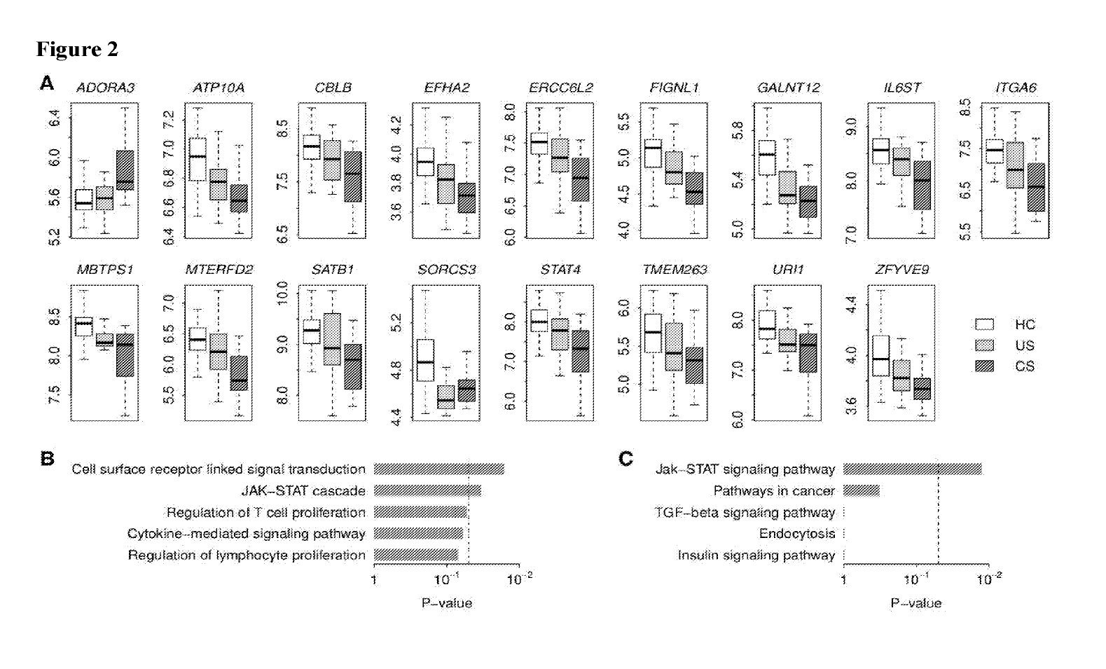

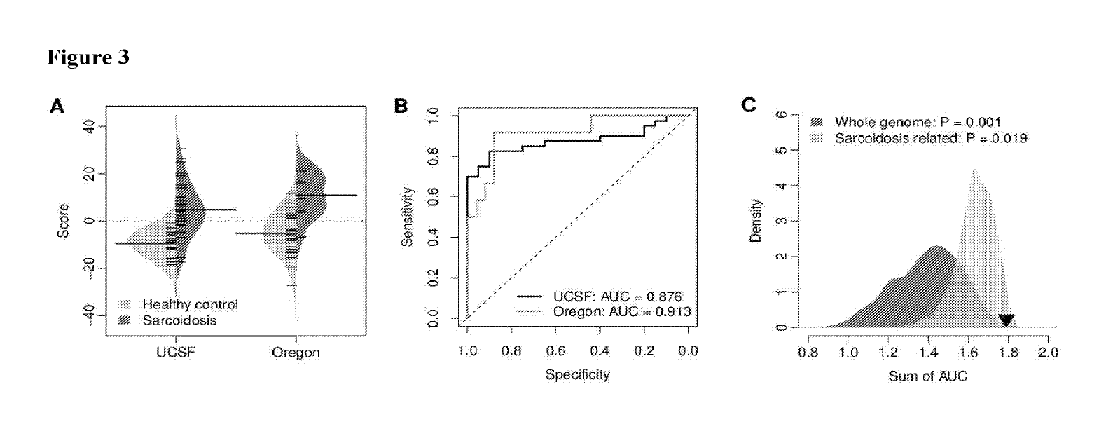

Results are shown in FIGS. 1-4 and Table 1. FIG. 1 shows the 8-miRNA signatures. The eight miRNAs were differentially expressed with the severity of sarcoidosis. Y-axis indicates the miRNA expression level. HC: healthy controls; US: uncomplicated sarcoidosis; CS: complicated sarcoidosis. FIG. 2 shows the 17-gene signature. (A) The 17 protein-coding genes were differentially expressed with the severity of sarcoidosis. Y-axis indicates the gene expression level. HC: healthy controls; US: uncomplicated sarcoidosis; CS: complicated sarcoidosis. (B) The top five GO biological process terms associated with the 17-gene signature. (C) The top five KEGG pathway terms associated with the 17-gene signature. The P-values in panel B and C were calculated by Fisher's exact test. The dash line denotes the significance level of 0.05. FIG. 3 shows the performance of the 17-gene signature in the validation cohorts. (A) The 17-gene signature based severity score differentiates the sarcoidosis patients from the healthy controls in the UCSF and Oregon cohorts. The violin plot indicates the distribution of the severity score in each category. (B) The ROC curves of the 17-gene signature in classifying the subjects in the UCSF and Oregon cohorts. (C) Superior predictive power of the 17-gene signature compared with random gene set. The dark grey area shows the distribution of the sum of AUC (both the validation cohorts) for the 1,000 resampled gene signatures (with the identical size as the 17-gene signature) randomly picked up from human genome. The light grey area shows the distribution of the sum of AUC for the 1,000 resampled gene signatures randomly selected from the pool of the sarcoidosis related genes. The black triangle stands for the sum of AUC of the 17-gene signature. Right-tailed P-values of the sampling distribution were calculated.

This example indicates that PBMC gene expression is useful in diagnosis of sarcoidosis, and in the identification of patients with complicated sarcoidosis.

TABLE 1
Sarcoidosis related miRNA and target gene pairs

| miRNA | Target gene | misSVR score | ρ | Adjsuted P |
|---|---|---|---|---|
| hsa-miR-23a | EFHA2 | −1.260 | −0.589 | 3.42 × 10⁻⁵ |
| hsa-miR-23a | GALNT12 | −1.264 | −0.480 | 1.78 × 10⁻³ |
| hsa-miR-23a | SATB1 | −1.342 | −0.454 | 3.75 × 10⁻³ |
| hsa-miR-23a | STAT4 | −1.276 | −0.475 | 2.12 × 10⁻³ |
| hsa-miR-23a | TMEM263 | −1.201 | −0.459 | 3.26 × 10⁻³ |
| hsa-miR-23b | EFHA2 | −1.260 | −0.531 | 3.49 × 10⁻⁴ |
| hsa-miR-23b | GALNT12 | −1.264 | −0.438 | 5.75 × 10⁻³ |
| hsa-miR-30c | ITGA6 | −1.203 | −0.433 | 6.46 × 10⁻³ |
| hsa-miR-93 | FIGNL1 | −1.231 | −0.463 | 2.91 × 10⁻³ |
| hsa-miR-93 | MBTPS1 | −1.228 | −0.533 | 3.23 × 10⁻⁴ |
| hsa-miR-93 | MTERFD2 | −1.231 | −0.540 | 2.51 × 10⁻⁴ |
| hsa-miR-93 | URI1 | −1.273 | −0.468 | 2.57 × 10⁻³ |
| hsa-miR-93 | ZFYVE9 | −1.332 | −0.511 | 7.03 × 10⁻⁴ |
| hsa-miR-143 | ATP10A | −1.333 | −0.443 | 5.05 × 10⁻³ |
| hsa-miR-185 | SORCS3 | −1.215 | −0.437 | 5.79 × 10⁻³ |
| hsa-miR-196a* | ADORA3 | −1.270 | −0.472 | 2.30 × 10⁻³ |
| hsa-miR-223 | CBLB | −1.252 | −0.447 | 4.50 × 10⁻³ |
| hsa-miR-223 | ERCC6L2 | −1.296 | −0.446 | 4.63 × 10⁻³ |
| hsa-miR-223 | IL6ST | −1.340 | −0.432 | 6.53 × 10⁻³ |

Note-ρ is the Spearman′s rank correlation coefficient. P-values were calculated by Spearman′s rank correlation test between miRNA and target gene expression levels and adjusted by Benjamini & Hochberg procedure.
Severity score:
$S = {\sum }_{i = 1}^{n} {W}_{i} ( {e}_{i} - {μ}_{i} ) / {τ}_{i}$ Wᵢ = 1, if up-regulated in sarcoidosis
Wᵢ = −1, if down-regulated in sarcoidosis
S is the risk score of the patient; n is the number of genes in the gene signature;
Wᵢ denotes the weight of gene i, which indicates the direction of deregulation for gene i (1 or −1);
eᵢ denotes the expression level of gene i and μᵢ and τᵢ are the mean and standard deviation of the gene expression values for gene i across all samples, respectively.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method of identifying miRNA expression, comprising:
assaying a sample from a subject for the presence of altered expression of one or more miRNAs selected from the group consisting of miR-23a, miR-23b, miR-30c, miR-93, miR-143, miR-185, miR-196a, and miR-223.

2. A method of identifying gene expression, comprising:
assaying a sample from a subject for the presence of altered gene expression of one or more genes selected from the group consisting of ADORA3, ATP10A, CBLB, EFHA2, ERCC6L2, FIGNL1, GALNT12, IL6ST, ITGA6, MBTPS1, MTERFD2, SATB1, SORCS3, STAT4, TMEM263, UR11, and ZFYVE9.

3. The method of claim 1, wherein said method further comprises the step of
assaying said sample for the presence of altered gene expression of one or more genes selected from the group consisting ofADORA3, ATP10A, CBLB, EFHA2, ERCC6L2, FIGNL1, GALNT12, IL6ST, ITGA6, MBTPS1, MTERFD2, SATB1, SORCS3, STAT4, TMEM263, UR11, and ZFYVE9.

4. The method of claim 2, further comprising the step of calculating a diagnostic score for said sample based on the expression of said one or more genes.

5. The method of claim 4, wherein a higher positive number score is indicative of said subject having or being at risk for complicated sarcoidosis.

6. The method of claim 1, wherein said at least one gene or miRNA is at least 5 of said miRNAs.

7. The method of claim 1, wherein said at least one gene or miRNA is at least 10 of said miRNAs.

8. The method of claim 1, wherein said at least one gene or miRNA is all of said miRNAs.

9. The method of claim 1, wherein the sample is selected from the group consisting of tissue, blood, plasma, serum, and lung cells.

10. The method of claim 1, wherein said detecting comprises forming a complex between said genes and a nucleic acid primer, probe, or pair of primers that specifically bind to said genes.

11-18. (canceled)

19. A kit, comprising: reagents for detecting altered expression levels of two or more miRNAs selected from the group consisting of miR-23a, miR-23b, miR-30c, miR-93, miR-143, miR-185, miR-196a, and miR-223 and/or two or more genes selected from the group consisting of ADORA3, ATP10A, CBLB, EFHA2, ERCC6L2, FIGNL1, GALNT12, IL6ST, ITGA6, MBTPS1, MTERFD2, SATB1, SORCS3, STAT4, TMEM263, UR11, and ZFYVE9.

20. The kit of claim 19, wherein said reagents are at least two nucleic acid primers or probes that specifically hybridizes to said two or more genes or miRNAs.

21. The kit of claim 19, wherein said nucleic acid primers or probes are at least 8 nucleic acids in length.

22. The kit of claim 19, wherein said nucleic acid primers or probes are at least 10 nucleic acids in length.

23. The kit of claim 19, wherein said nucleic acid primers or probes are at least 20 nucleic acids in length.

24. The kit of claim 19, wherein said two or more of said genes or miRNAs is five or more of said genes and/or miRNAs.

25. The kit of claim 19, wherein said two or more of said genes or miRNAs is 10 or more of said genes and/or miRNAs.

26. The kit of claim 19, wherein said two or more of said genes or miRNAs is all of said genes and/or miRNAs.

27-33. (canceled)
